(19) United States
(12) Patent Application Publication
(10) Pub. No.: US 2007/0102009 A1
(43) Pub. Date: May 10, 2007

(54) Method and device for snoring management

(76) Inventors: Thomas K.S. Wong, Hong Kong (CN); Joanne W.Y. Chung, Hong Kong (CN); K.L. Fan, Hong Kong (CN); W.L. Chow, Hong Kong (CN); X.M. Tao, Hong Kong (CN); Alice S.L. Ng, Hong Kong (CN)

Correspondence Address: KNOBBE MARTENS OLSON & BEAR LLP, 2040 MAIN STREET, FOURTEENTH FLOOR, IRVINE, CA, 92614, US

(21) Appl. No.: 11/267,427

(22) Filed: Nov. 4, 2005

(51) Int. Cl. A61B 19/00

(52) U.S. Cl. 128/898; 602/902

(57) ABSTRACT

An anti-snoring device and method for stopping a person from snoring. The device comprises a casing that contains a microcontrol unit and is adapted to be wearable on a person's limb. The casing is connected to a belt having an inner surface and an outer surface, and the belt comprises two electrically conductive wires. Each electrically conductive wire has one end being exposed to the inner surface of the belt and the other end leading from the casing. A microphone is also included into the device for detecting snoring sounds and communicating with the microcontrol unit. The device can detect snoring sound and send an electrical stimulation to an acupuncture point of the person to reduce or eliminate snoring.

### BACKGROUND

1. Field of the Invention

The field of the invention relates to snore management.

2. Description of the Related Technology

Snoring is a noise generated by vibration of the air conduction tube at the level of the throat. Snoring occurs when, during sleep, there is a loss of muscle tone in the muscles of the throat, which are needed to keep the airway open. The loss in muscle tone narrows the air passages leading to inadequate levels of airflow into the lungs. This drop in airflow is detected and the body's reflex automatically increases the body's efforts to breathe. These increased efforts produce greater degrees of suction in the air passage which then in turn causes the walls of the throat to vibrate and flutter. Most commonly, snoring is generated when the soft palate flutters. This fluttering leads to pressure oscillations within the airway and sounds are then generated when other structures in and around the airway are vibrated.

Snoring can affect sleep quality. Snorers and their bed partners or other family members are all influenced. About one-third of older adults snore. The common management methods of snores are weight control, changing sleeping positions, applying anti-snoring pills, oral appliances, nasal strips, and uvulopalatophryngoplasty (UPPP, a surgery method to remove soft tissues at the back of the throat and soft palate).

Example of the devices that attempt to stop a person's snoring by changing sleeping positions can be found in U.S. Pat. No. 5,458,105 to Taylor, and in http://www.meridianhk.com.hk/snore_stopper_main.html to a commercial product sold under the name “Snore Stopper™.”

The '105 patent to Taylor describes a device to be worn on a person's wrist that detects snoring and then induces a vibratory motion to the person's wrist. The Taylor's device composes of a housing that begins to vibrate when it detects the snoring exceeding a certain predetermined level. The vibrating motion of the housing is intended to cause the person to roll over or change sleeping positions, which tends to cause the snoring to stop.

The “Snore Stopper™,” also wearable on a snorer's wrist, detects snoring and then sends an electrical pulse to stimulate nerves on the snorer's skin. The brain then senses this nerve stimulation and reminds or trains the snorer to change his or her sleeping posture, thereby reducing snoring. The device composes of a housing and operates via an external pair of self-adhesive electrode pads. The electrode pads need maintenance and replacements after being used for 30 nights continuously. Users are cautioned against wearing without the self-adhesive electrode pads being stuck to the housing, and against wearing on the underside of the wrist

The compliances and the efficacies of the above-mentioned snore management methods vary among the snorers. Many snorers, therefore, currently prefer to be untreated. Thus, there is a need for a safe, non-invasive, user-friendly, less cumbersome and cost-effective approach for the management of snoring.

### SUMMARY OF CERTAIN EMBODIMENTS

Accordingly, one object of the present invention is to provide a better management of snoring by applying the theory of Traditional Chinese Medicine (TCM) acupoint, bio-signal processing algorithms and smart textile technology.

Another object of the present invention is to provide a method and a device for reducing or eliminating snoring in a person by sending an electronic pulse to an acupoint of the person when snoring occurs.

In order to achieve these and other objects of the invention, an anti-snoring device is provided that can promptly detect the occurrence of snoring and release an electrical stimulation to an acupoint of the snorer (e.g., the Lieque acupoint, LU 7) upon detection of a snoring event.

An embodiment provides for an anti-snoring device that comprises a casing that contains a microcontrol unit (MCU) and is adapted to be wearable on a person's limb; a belt that has an inner surface and an outer surface and is adapted to connect to the casing and to secure the casing on the person's limb; two electrically conductive wires (electrodes) that lead from the casing and have one end being exposed to the inner surface of the belt; and a microphone that can detect a series of discrete snoring sounds and communicate with the MCU.

Another embodiment provides for a method for management of snoring. The method features delivering an electrical stimulation to a snorer's acupoint when a snoring event is detected. In one aspect of the invention, the method comprises detecting a series of sounds in an environment and communicating the sounds to a MCU, determining whether the sounds exceed a pre-determined decibel level, or whether a discrete snoring sound pattern exists, and delivering an electrical stimulation to an acupoint after the pre-determined level or threshold is exceeded, to reduce or eliminate the snoring. A specific acupoint along the Lung Meridian line may be chosen for effectively managing snoring. For example, the Lung Acupoint Number 7, or LU 7, or Lie Que (Chinese Character: “ [symbol] ”) can be chosen to be the target for stimulation. While not to be bounded by the theory, the LU 7 point is believed to connect to the human throat and thus by stimulating it the symptom of snoring will be reduced or eliminated.

The details of one or more embodiments of the invention are set forth in the accompanying description below. Other advantages, features, and objects of the invention will be apparent from the detailed description and the claims.

### BRIEF DESCRIPTION OF THE DRAWINGS

FIG. 1 illustrates Lung Acupoints and LU 7 (Lieque) (Chinese Character: “ [symbol] ”)

FIG. 2 is a perspective view of one embodiment of an anti-snoring device.

FIG. 3 is a perspective view of the anti-snoring device shown in FIG. 2 with the top casing removed.

FIG. 4 is a perspective view of the anti-snoring device shown in FIG. 2 with the bottom casing removed.

FIG. 5 is a block diagram of one embodiment of an anti-snoring device.

FIG. 6 is a block diagram of another embodiment of an anti-snoring device.

### DETAILED DESCRIPTION OF CERTAIN EMBODIMENTS

The invention applies the Traditional Chinese Medicine acupoint theory, bio-signal processing algorithms and smart textile technology to provide a method and device for snore management.

In ancient Chinese medicine there are 365 acupoints that are mapped to 14 major meridian lines: one meridian for each of the 12 inner organs, one meridian along the spine (the governing vessel), and another along the midline of the abdomen (the conception vessel).

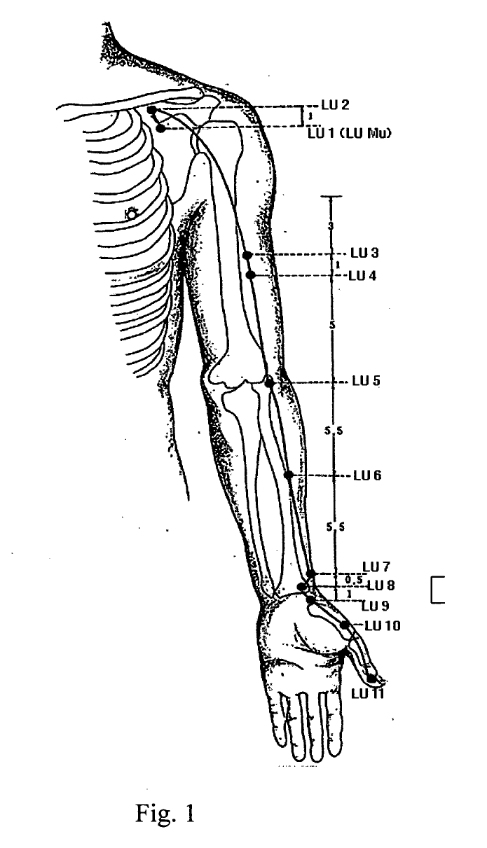

FIG. 1 shows the Lung Acupoints L1-L11. Each lung point has its function in modulating internal body organs and thus achieves the purpose of treating disorders. (see http://www.acuxo.com/meridianPictures.asp?point=LU11&meridian=Lung). In many individuals, the acupoint LU7, or Lieque, is located about 1.5 inches above the wrist, about 0.2-0.3 inches deep, on the inside of the arm. The point is in the depression superior to the styloid process of the radius. This point in traditional Chinese Medicine is used to treat several disorders of the upper body, including neck stiffness, cough, asthma, and sore throat. While not to be bounded by the theory, it is believed that the LU7 point connects to airway muscle and stimulation of it may normalize airway muscle tone and thus stop snoring symptoms.

In one embodiment the invention provides a method and device for reducing snoring symptoms. The invention features detecting a series of sounds and communicating the sounds to a microcontrol unit (MCU), determining whether a snoring sound pattern exists, or whether the sound exceeds a pre-determined threshold, and delivering an electrical stimulation to an acupoint of a user to reduce or eliminate the snoring.

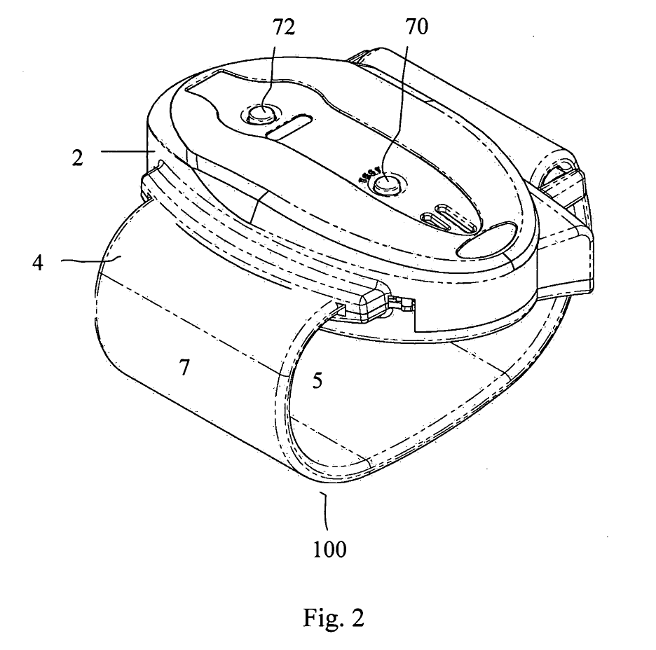

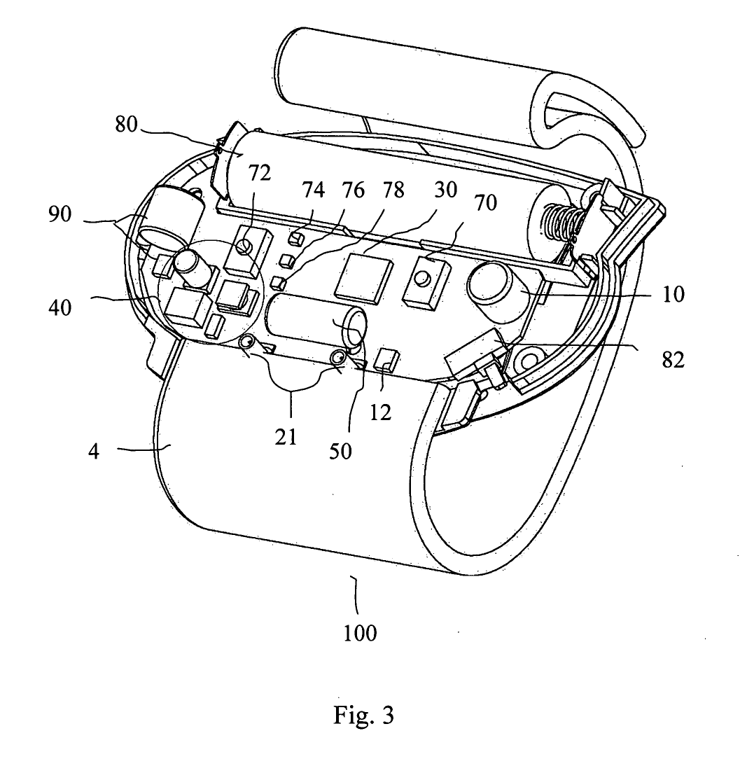

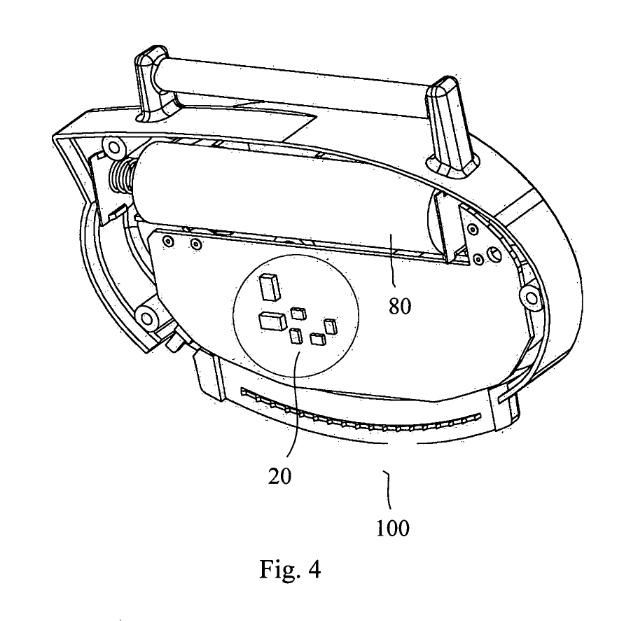

Referring to FIGS. 2 through 5, one embodiment of an anti-snoring device 100 comprises a casing 2, a belt 4, two electrically conductive wires (electrodes) 6, and an electronic microphone 10. The casing 2 comprises a microcontrol unit (MCU) 30 and has a configuration that is wearable on a person's wrist or other limb. The casing 2 is preferably made of ABS plastic. The belt 4 has an inner surface 5 and an outer surface 7 and is adapted to connect to the casing 2. The two electrically conductive wires (electrodes) 6 lead from said casing 2, and of which one end is exposed on the inner surface 5 of said belt 4 (electrodes not shown in FIGS. 2 and 3).

The belt 4 may be made of resilient materials, such as the plastic or rubber or composite materials for providing a suitable resilience to wrap around the limb portion of users. Preferably, the belt 4 is elastic fabric made of 95% polyester and 5% Lycra (a trademark used for a brand of spandex). The elastic fabric may be a plain weft rib knitted structure, knitted by using a tubular knitting machine. In some embodiments, the diameter of the fiber may be about 10 microns, and the knitting structure of the fabric may be about 16 courses by about 20 wales per centimeter.

If desired, fasteners, such as VELCRO®-type fasteners, and buckles may be adapted as accessories of the belt 4. For instance, the belt 4 may contain two sections, with only one section having the two electrically conductive wires (electrodes) 6. The section having the two electrically conductive wires (electrodes) 6 may be made adjustable in length by using the fastener, and may be connected to the other section of the belt 4 by the buckles. The fastener may be sewed onto the fabric by lock stitches, and the buckles sewed using hand-stitches.

In one aspect of invention, the two electrically conductive wires (electrodes) 6 are embedded inside said belt 4 with one end being exposed to the inner surface 5 of said belt 4. The electrically conductive wires (electrodes) 6 may be made of any conductive material, including metal. In one aspect of the invention, the electrically conductive wires (electrodes) 6 are made of 100% stainless steel, or 100% silver coated with 100% polyester. Preferably, the electrically conductive wires (electrodes) 6 are woven onto the elastic fabric of the belt 4 to form a smart conductive fabric. The two electrically conductive wires (electrodes) 6 may be sewn onto the fabric to make a conductive fabric using an embroidery machine and cotton sewing threads. The width of the embroidery line may be about 2 mm. The two electrically conductive wires (electrodes) 6 may release electrical stimulations upon the skin surface of a user. To stimulate a Lung Acupoint, one of the electrodes 6 is preferably located at about the target Acupoint, such as LU7, and the other electrode positioned along the Lung Meridian.

The electronic microphone 10 may be housed within, or physically separate from, said casing 2. The microphone 10 is for detecting a series of discrete snoring sounds and communicating with a MCU 30.

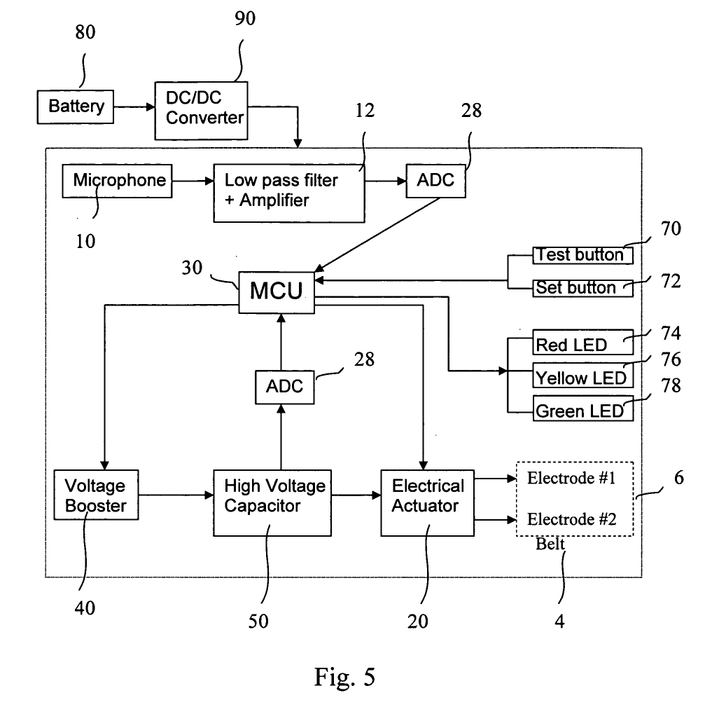

Referring to FIG. 5, the microphone 10 is housed within the casing 2 and wire-connected to the MCU 30. The microphone 10 detects a series of sounds, which are amplified and filtered by a microphone amplifier 12 (e.g., an LM321 operational amplifier). The output of the microphone amplifier 12 is connected to microcontroller (MCU) 30 (e.g., an ATMEGA48 from ATMEL). A MCU 30 internal Analog to Digital Converter (ADC) 28 (10 bit) converts an analog sound signal from the microphone amplifier 12 to digital data. The MCU 30 generates a high-speed pulse width modulation (PWM) signal, which is transmitted to a voltage booster 40 (Resistor, Inductor, Capacitor circuit) to charge a high voltage capacitor 50 (e.g., about 100V) up to a certain voltage level (from about 30 V to about 80V, depending on the chosen electrical stimulation level). An electrical actuator 20 having two PNP and two NPN transistors may be provided to control the energy flow from the high voltage capacitor 50 to a user's skin. The output terminal 21 of the electrical actuator 20 is connected (e.g., mechanically clamped) to one end of the electrically conductive wires (electrodes) 6.

Button switches, such as the Set button 72 and Test button 70, and LEDs, such as red LED 74, yellow LED 76 and green LED 78, may also be provided to directly connect to the MCU 30 as a user interface.

The MCU 30 determines whether a snoring sound pattern exists or not. It determines a rise of a sound signal level within a certain period. If the peak of the sound signal level exceeds a predetermined decibel level (e.g., about 65 dB), the MCU 30 will treat the sound signal as activation of a discrete snoring sound pattern. The word “discrete” refers to the peak of each individual snoring signal of the pattern. When the sound signal level decreases and returns to a lower level for a certain period after the peak, the MCU 30 treats the sound signal as a complete cycle and count it as one detected snoring signal or cycle. For a detected sound signal that does not meet the above-mentioned criteria, the MCU 30 will treat the signal as a noise signal and will not count the detected noise as a complete snoring cycle. Therefore, if the anti-snoring device of the present invention is placed in a noisy environment, the red LED may be always on but any noise will not be treated as a snoring sound pattern.

After a series of sounds detected by the microphone 10 reaches a predetermined decibel level or threshold as determined by the MCU 30, an electrical pulse will be released by the actuator 20. The pre-determined decibel level or threshold is adjustable. In one aspect of the invention, the anti-snoring device releases an electrical stimulation after a certain number of snoring cycles (e.g. about 3 cycles) are detected within a certain period of time (e.g. about 10 sec). The MCU will reset its counter to zero after a certain period of time (e.g. about 10 sec).

The term “decibel” is a unit of measurement of the loudness or strength of a signal. It is a relative measurement derived from two signal levels: a reference input level and an observed output level. A decibel is the logarithm of the ratio of the two levels. One bel is when the output signal is 10× that of the input, and one decibel is 1/10th of a bel.

The intensity of electrical stimulations may be adjusted or controlled by the “SET button 72. By pressing the SET button 72, a control signal is sent to the voltage booster 40 and the energy is stored in the high voltage capacitor 50. In some embodiments, the high voltage capacitor may be a 100V capacitor 50. The stimulation intensity is adjusted by controlling the voltage stored at the high voltage capacitor 50. When the capacitor voltage is charged to the preset voltage level (read by said MCU 30 through the ADC 28), the MCU 30 will stop the voltage booster 40 from further charging the capacitor 50 and the capacitor 50 is now ready to release energy to the electrical actuator 20. The actuator 20 is composed of an electric circuit, which is able to release electrical pulses in both directions to form biphasic pulses (current flow from electrode No. 1 to electrode No. 2 or from electrode No. 2 to electrode No. 1). The electrical actuator 20 delivers small electrical pulses to the skin surface via the electrically conductive wires (electrodes Nos. 1 and 2) 6, which are embedded in the belt 4. In one embodiment, no conductive gel is applied to the user for the electrical stimulation.

The LEDs 74, 76 and 78 of the device 100 may also be used to display an event log. For example, the device 100 may be programmed so as to indicate the number of snoring events as follows, for example,: Red light: number of snoring events is more than 10; yellow light: number of snoring events is less than 10 but more than 5; green light: number of snoring events is less than 5. This record may be cleared after pressing either the SET button 72 or TEST button 70 and a new cycle will start.

The device 100 may be powered by a single AAA size 1.5 V battery 80. A DC/DC converter 90 (e.g, ST5R50M from STMicroelectronics) may be used to step up voltage up to 5V for the whole circuit.

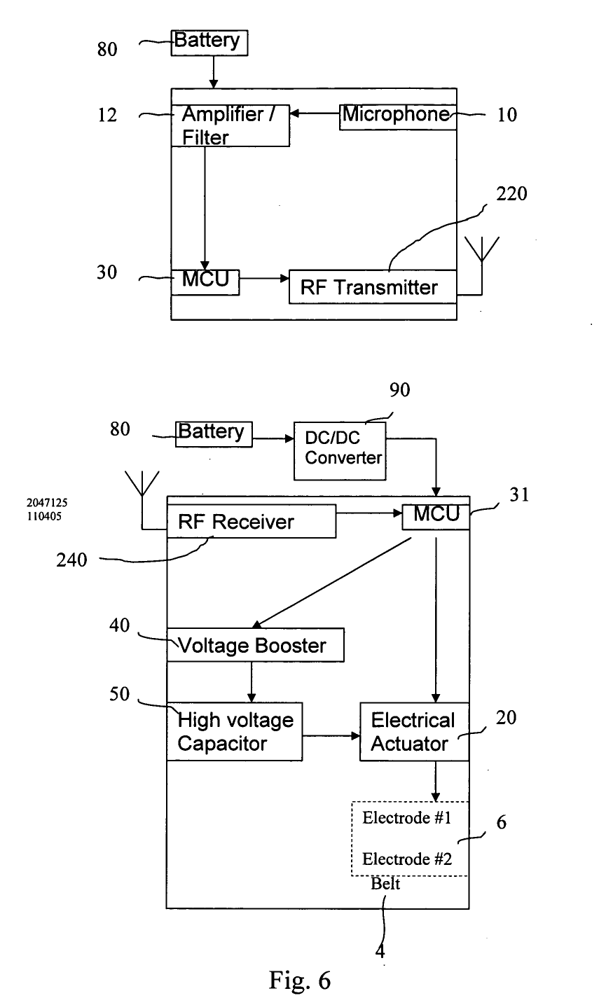

FIG. 6 illustrates another embodiment of the invention, where the electronic microphone 10 is physically separated from the casing 2. In this embodiment, the invention further comprises two additional electronic components, RF transmitter 220 and RF receiver 240, to enable a wireless communication between the microphone 10 and the MCU 31 within the casing 2. In this embodiment, the microphone 10 detects a series of sounds in an environment and communicates the series of sounds to said MCU 30 that is integrated with the microphone 10. The MCU 30 determines whether the series of sounds exceeds a predetermined decibel threshold, and/or whether a snoring sound pattern exists, and further communicates the series of sounds to another MCU 31 that is located within the casing 2, where the communication is performed by the RF transmitter 220 the RF receiver 240.

RF transmitter 220 and RF receiver 240 may be commercially available. RF transmitter/Receiver as illustrated here, although other wireless means based on WiFi, Blue Tooth and other short distance communication technologies such as Ultra-Wideband (UWB) technology may also be used.

Snorers should wear the anti-snoring device of the invention during sleep. The anti-snoring device helps the snorers identify snoring and to provide instant responses and actions for minimizing repetitive episodes of snoring.

To illustrate usage of the invention, the exposed ends (terminals) of the two electrically conductive wires (electrodes) 6 on the belt 4 are to be positioned at the Lieque acupoint (LU 7), and the belt 4 is then tightened to secure the electrodes 6 at the LU 7 location so that the terminals of said electrodes 6 will not slide off the acupoint while moving about in sleep.

The power switch 82 may be shifted to an “ON” position to turn on the device. In one embodiment, when the device is turned on, the Green LED 78 will be on. The intensity of electrical stimulation may be set at a desired level by pressing the SET button 72, or the device is ready for use by simply pressing the TEST button 70. If the TEST button 70 is pressed, all the LEDs will be turned off and the device is in operation using the previously stored electric stimulation level.

In one embodiment, to set the level of electrical stimulation, a user presses the “SET” button 72. By continuously pressing the “SET” button 72, the LEDs will be lighted in a sequence of green, yellow and red, which represents low, medium and high stimulation intensity, respectively. For example, if the “SET” button 72 is pressed, the green LED 78 will be on to indicate the device is at level 1 (weakest). By pressing the “SET” button 72 again, the yellow LED 76 will be on to indicate level 2. By pressing the “SET” button 72 again, the red LED 74 will be on to indicate level 3 (strongest). It will return to level 1 if the “SET” button 72 is pressed further. The LEDs will automatically switch off after a particular setting is selected and the device is ready to use.

To experience the level of electrical stimulation, a user may press the “TEST” button 70. The user should choose the highest, tolerable intensity of electrical stimulation to achieve a maximal, anti-snoring effect. After testing the intensity of the electric stimulation, the LEDs will be off and the device is in operation and the electric stimulation level selected is automatically stored.

A user who cannot experience or feel an electrical stimulation may need to use the highest level and/or adjust said belt 4 tension to ensure said electrodes 6 have good contact with the user's skin. The battery may also need to be replaced if an electrical stimulation is still not experienced.

When in operation mode, the anti-snoring device of this invention detects a series of sounds in the environment. The red LED 74 will be on when a sound level exceeds a certain predetermined level (e.g., 65 dB) to indicate the device is in operation.

In one embodiment, once the device is in operation, it may count the number of activations (number of times it sends an electric pulse to the user's acupoint). The device will show the number of activations through the LEDs. This counter will be reset to zero after the user presses said “SET button” 72 again.

Any patents or publications mentioned in this specification are indicative of the levels of those skilled in the art to which the invention pertains. Further, these patents and publications are incorporated by reference to the same extent as if each individual publication was specifically and individually indicated to be incorporated by reference.

While there have been described and pointed out fundamental novel features of the invention as applied to a preferred embodiment thereof, it will be understood that various omissions and substitutions and changes, in the form and details of the embodiments illustrated, may be made by those skilled in the art without departing form the spirit of the invention.

What is claimed is:

1. An anti-snoring device comprising:
(a) a casing, comprising a microcontrol unit and adapted to be wearable on a person's limb;
(b) a belt for securing said casing on said person's limb, having an inner surface and an outer surface and adapted to connect to the casing;
(c) two electrically conductive wires leading from said casing, one end of each wire being exposed on said inner surface of said belt; and
(d) a microphone for detecting sounds and being in communication with said microcontrol unit.

2. The anti-snoring device according to claim 1, wherein said microphone is integrated into the casing.

3. The anti-snoring device according to claim 1, wherein said microphone is physically separate from said casing.

4. The anti-snoring device according to claim 1, wherein said electrically conductive wires are embedded in the belt.

5. The anti-snoring device according to claim 1, wherein said belt is fabric and said electrically conductive wires are woven into the fabric.

6. The anti-snoring device according to claim 1, wherein said electrically conductive wires are made of polyester coated with a metal or a mixture of two or more metals.

7. The anti-snoring device according to claim 6, wherein said polyester is coated with silver.

8. The anti-snoring device according to claim 1, wherein said electrically conductive wires are made of stainless steel.

9. The anti-snoring device according to claim 1, wherein said casing further comprises:
(a) a high voltage capacitor for storing electrical charges;
(b) a voltage booster for charging said high voltage capacitor; and
(c) an electrical actuator for releasing an electrical pulse.

10. The anti-snoring device according to claim 1 further comprising a set button for presetting said electrical pulse at a preset level of intensity.

11. The anti-snoring device according to claim 1 further comprising a LED for displaying said preset level of intensity, or displaying events of releasing said electrical pulse, or both.

12. The anti-snoring device according to claim 1 further comprising a test button for testing said preset level of intensity of said electrical pulse.

13. The anti-snoring device according to claim 7 further comprising a set button for presetting said electrical pulse at a preset level of intensity.

14. A method for reducing or eliminating the snoring of a person comprising the steps of:
(a) detecting a series of sounds in an environment and communicating said sounds to a microcontrol unit,
(b) determining whether said series of sounds exceeds a pre-determined threshold;
(c) delivering an electrical stimulation after said threshold is exceeded, to an acupoint of said person for reducing or eliminating the snoring.

15. The method according to claim 14, wherein the acupoint is a Lung Acupoint.

16. The method according to claim 15, wherein the Lung Acupoint is LU7(Lieque).

17. The method according to claim 14, wherein said threshold can be adjustable.

18. The method according to claim 14, wherein said electrical stimulation is delivered after said threshold is exceeded three times.

19. The method according to claim 14, wherein in step (b) further comprising communicating said sound level by a wireless means.
